(19) United States
(12) Patent Application Publication
(10) Pub. No.: US 2014/0088588 A1
(43) Pub. Date: Mar. 27, 2014

(54) SYSTEMS AND METHODS FOR CONTROLLING ENERGY APPLICATION

(71) Applicant: Boston Scientific Scimed, Inc., Maple Grove, MN (US)

(72) Inventor: Jerry JARRARD, Longmont, CO (US)

(73) Assignee: Boston Scientific Scimed, Inc., Maple Grove, MN (US)

(21) Appl. No.: 14/032,754

(22) Filed: Sep. 20, 2013

Related U.S. Application Data
(60) Provisional application No. 61/705,839, filed on Sep. 26, 2012.

(51) Int. Cl. A61B 18/12; A61B 18/14

(52) U.S. Cl. CPC A61B 18/1233; A61B 18/1482
USPC 606/34; 606/41

(57) ABSTRACT

Energy delivery systems and methods for treating tissue are disclosed that may include an energy generator, a cooled electrode device, and a controller connected to the energy generator. The controller may include a processor and may be configured to control power output by the cooled electrode device based on a measured impedance level of tissue at a target treatment site.

### CROSS-REFERENCE TO RELATED APPLICATION

This patent application claims the benefits of priority under 35 U.S.C. §119 to U.S. Provisional Patent Application No. 61/705,839, filed Sep. 26, 2012, the entirety of which is incorporated herein by reference.

### TECHNICAL FIELD

Embodiments of the present disclosure relate generally to devices and methods for treating tissue in a cavity or passageway of a body. More particularly, embodiments of the present disclosure relate to devices and methods for treating tissue in an airway of a body, among other things.

### BACKGROUND

The anatomy of a lung includes multiple airways. As a result of certain genetic and/or environmental conditions, an airway may become fully or partially obstructed, resulting in an airway disease such as emphysema, bronchitis, chronic obstructive pulmonary disease (COPD), and asthma. Certain obstructive airway diseases, including, but not limited to, COPD and asthma, are reversible. Treatments have accordingly been designed in order to reverse the obstruction of airways caused by these diseases.

One treatment option includes management of the obstructive airway diseases via pharmaceuticals. For example, in a patient with asthma, inflammation and swelling of the airways may be reversed through the use of short-acting bronchodilators, long-acting bronchodilators, and/or anti-inflammatories. Pharmaceuticals, however, are not always a desirable treatment option because in many cases they do not produce permanent results, or patients are resistant to such treatments or simply non-compliant when it comes to taking their prescribed medications.

Accordingly, more durable/longer-lasting and effective treatment options have been developed in the form of energy delivery systems for reversing obstruction of airways. Such systems may be designed to contact an airway of a lung to deliver energy at a desired intensity for a period of time that allows for the airway tissue (e.g., airway smooth muscle, nerve tissue, etc.) to be altered and/or ablated. These systems typically monitor and/or control energy delivery to the airway tissue as a result of sensed temperature at an electrode/tissue interface. That is, a determination of appropriate treatment is made as a function of measured temperature at the electrode/tissue interface. Temperature monitoring at the electrode/tissue interface, however, is not always an accurate measure of tissue temperature below the tissue surface, particularly when cooling is involved. During treatment of tissue for reversing obstruction of airways, it may be beneficial to accurately measure the tissue temperature of the entire altered and/or ablated volume of tissue in order to determine the appropriate amount of energy delivery for treatment of the airway. There is accordingly a need for an energy delivery system that enables control of energy based on accurate temperature measurements of the altered and/or ablated volume of tissue in an airway or measurement of a variable indicative of such tissue temperatures.

### SUMMARY OF THE DISCLOSURE

Energy delivery systems and methods for treating tissue are disclosed in the present disclosure. Energy delivery systems may include an energy generator, a cooled electrode device, and a controller connected to the energy generator. The controller may include a processor and may be configured to control power output by the cooled electrode device based on a measured impedance level of tissue at a target treatment site (e.g., an initial impedance value).

Embodiments of the energy delivery systems may include one or more of the following features: the controller may be configured to control power output based on a second impedance level set in the controller (e.g., a set impedance value); the controller may be configured to calculate the second impedance level; the controller may be configured to calculate the second impedance level based on a percentage of the an initial impedance level measured at the target treatment site; the controller may be configured to calculate the second impedance level based on at least one of: a parameter of tissue at the target treatment site, a parameter of the cooled electrode device, a desired temperature range of tissue at the target treatment site, and a parameter of a pre-treatment energy output pulse; the controller may be configured to determine a temperature that correlates to the measured impedance level; and the cooled electrode device may include an internal portion for cooling the cooled electrode device when the cooled electrode device is in contact with tissue at the target treatment site.

Energy delivery systems are also disclosed that may include an energy delivery device including a cooled electrode device configured for connecting to an energy generator on a controller. The cooled electrode device may be configured to output power based on an initial impedance level of tissue at a targeted treatment site, and a second impedance level corresponding to a desired temperature of tissue at the targeted treatment site. The cooled electrode device may be configured to output power based on an application of the second impedance level to a PID (proportional, integral, derivative) algorithm, and the cooled electrode device may be configured to output power to tissue in a lung of an airway.

Methods for treating tissue may include determining an initial impedance level of tissue at a targeted treatment site with an energy delivery system comprising an energy generator, a cooled electrode device, and a controller including a processor; determining a second impedance level with the energy delivery system, wherein the second impedance level corresponds to a desired temperature of tissue at the targeted treatment site; and applying power to the tissue at the targeted treatment site through the cooled electrode device, wherein a power output level may be determined based on the second impedance level.

Methods for treating tissue may further include one or more of the following features: the tissue at the targeted treatment site may be located within an airway in a lung of a body; the controller may determine the second impedance level; the controller may determine the second impedance level based on a percentage of the initial impedance level; the controller may determine the second impedance level based on at least one of: a parameter of tissue at the target treatment site, a parameter of the cooled electrode device, a desired temperature range of tissue at the target treatment site, and a parameter of a pre-treatment energy output pulse; the controller may determine the power output level, which may include applying the second impedance level to a PID algorithm; repeating the step of determining the second impedance level throughout a cycle of treating tissue at the targeted treatment site; adjusting the power output level based the re-determined second impedance level; the targeted treatment site may be a first targeted treatment site, such that the method may include determining a second impedance level at a second treatment site, and applying power to the second treatment site based on the second impedance level determined at the second treatment site; and the step of cooling the tissue before, during, or after the step of applying power to the tissue.

Additional objects and advantages of the disclosure will be set forth in part in the description which follows, and in part will be obvious from the description, or may be learned by practice of the disclosure. The objects and advantages of the disclosure will be realized and attained by means of the elements and combinations particularly pointed out in the appended claims.

The accompanying drawings, which are incorporated in and constitute a part of this specification, illustrate several embodiments of the present disclosure and together with the description, serve to explain the principles of the invention.

### BRIEF DESCRIPTION OF THE DRAWINGS

FIG. 1 is a schematic view of airways within a lung.

FIG. 2A is a schematic view of a system for delivering energy to tissue within a cavity or passageway of a body according to a first embodiment of the present disclosure.

FIG. 2B is an enlarged view of a distal portion of a therapeutic energy delivery device, according to a first embodiment of the present disclosure.

FIG. 2C is an enlarged view of an electrode of the therapeutic energy delivery device of FIG. 2B.

FIG. 3A is a schematic view of an energy delivery device according to a second embodiment of the present disclosure.

FIGS. 3B-3C are enlarged views of a distal portion of the energy delivery device of FIG. 3A.

FIG. 4 is a flow diagram illustrating a procedure for controlling power during treatment according to an embodiment of the present disclosure.

### DESCRIPTION OF THE EMBODIMENTS

Reference will now be made in detail to exemplary embodiments of the present disclosure, examples of which are illustrated in the accompanying drawings. Wherever possible, the same reference numbers will be used throughout the drawings to refer to the same or like parts.

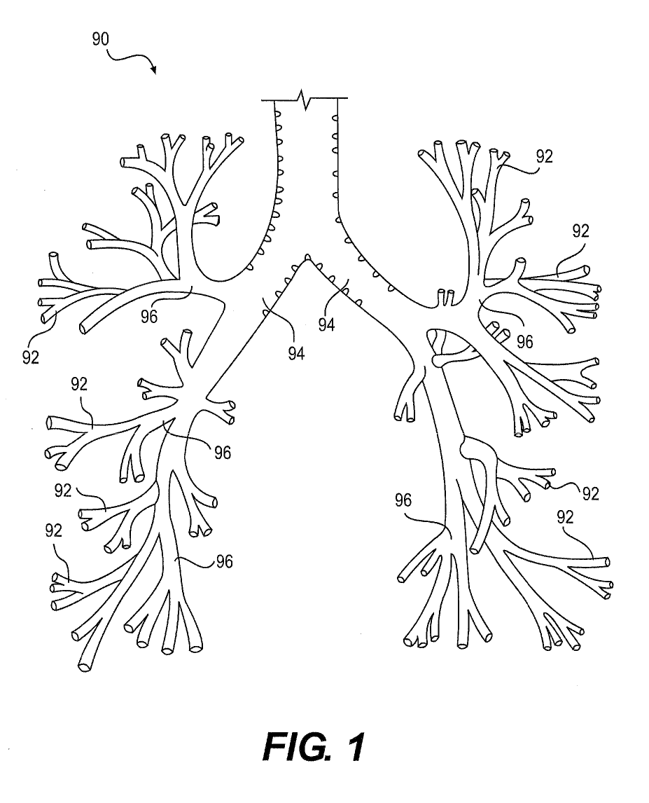

Embodiments of the present disclosure relate to devices and methods for controlling the application of energy to tissue within a wall or cavity of a body. More particularly, embodiments of the present disclosure relate to devices and methods for controlling the application of energy to tissue in the airway of a lung in order to treat reversible obstructive airway diseases including, but not limited to, COPD and asthma. Accordingly, devices of the present disclosure may be configured to navigate through tortuous passageways in the lungs, such as those illustrated in FIG. 1. Specifically, FIG. 1 illustrates a bronchial tree 90 having a right bronchi 94 and a left bronchi 94. Each of the right and left bronchi 94 includes a plurality of branches 96 with bronchioles 92 extending therefrom. It should be emphasized, however, that embodiments of the present disclosure may also be utilized in any procedure where heating of tissue is required, such as, for example cardiac ablation procedures, cancerous tumor ablations, etc.

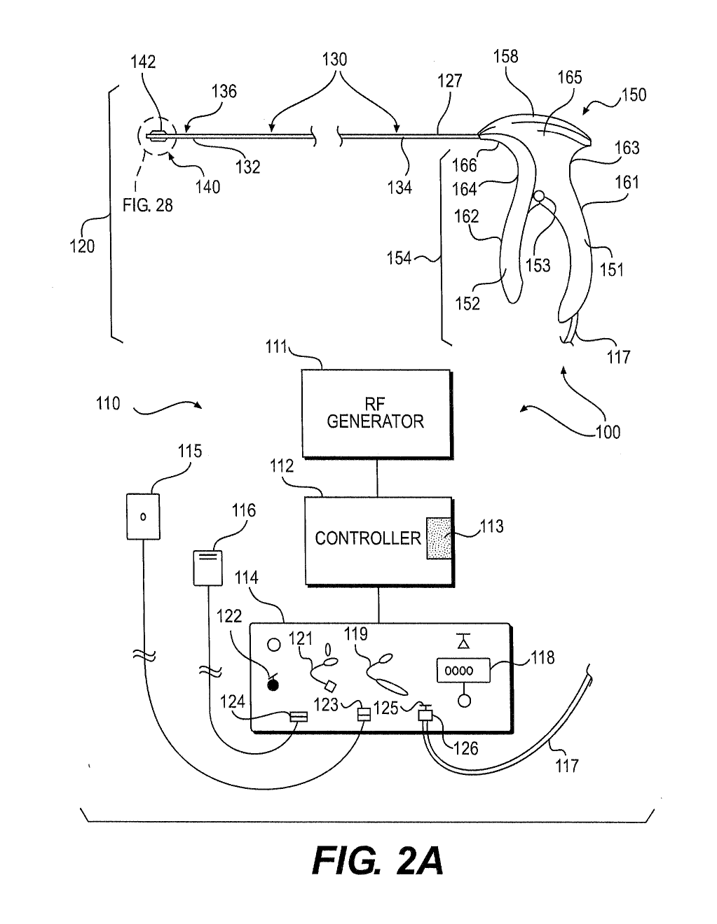

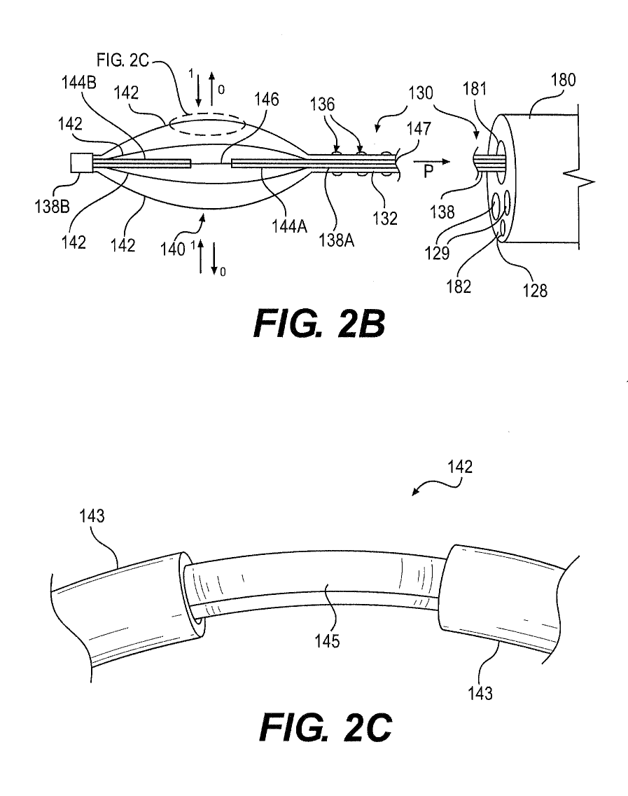

FIG. 2A illustrates a system for delivering energy 100, in accordance with a first embodiment of the present disclosure. The system may include and a control unit 110 and an energy delivery device 120. Control unit 110 may comprise a plurality of components, including, but not limited to, an energy generator 111, a controller 112, and a user interface 114. Energy generator 111 may be any suitable device configured to produce energy for heating and/or maintaining tissue in a desired temperature range. In one embodiment, for example, energy generator 111 may be an RF energy generator. The RF energy generator may be configured to emit energy at specific frequencies and for specific amounts of time in order to reverse obstruction in an airway of a lung.

In certain obstructive airway diseases, obstruction of an airway may occur as a result of narrowing due to airway smooth muscle contraction. Accordingly, in one embodiment, energy generator 111 may be configured to emit energy that reduces the ability of the smooth muscle to contract, increases the diameter of the airway by debulking, denaturing, and/or eliminating the smooth muscle or nerve tissue, and/or otherwise alters airway tissue or structures. That is, energy generator 111 may be configured to emit energy capable of ablating or killing smooth muscle cells or nerve tissue, preventing smooth muscle cells or nerve tissue from replicating, and/or eliminating smooth muscle or nerve tissue by damaging and/or destroying the smooth muscle or nerve tissue.

More particularly, energy generator 111 may be configured to generate energy with a wattage output sufficient to maintain a target tissue temperature in a range of about 60 degrees Celsius to about 80 degrees Celsius. In one embodiment, for example, energy generator may be configured to generate RF energy at a frequency of about 400 kHz to about 500 kHz and for treatment cycle durations of about 5 seconds to about 15 seconds per treatment cycle. Alternatively, the duration of each treatment cycle may be set to allow for delivery of energy to target tissue in a range of about 125 Joules of RF energy to about 150 Joules of energy. In one embodiment, for example, the duration of treatment for a monopolar electrode may be about 10 seconds to achieve a tissue temperature of approximately 65 degrees Celsius. In another embodiment, the duration of treatment for a bipolar electrode may be approximately 2 to 3 seconds to achieve a tissue temperature of approximately 65 degrees Celsius.

Energy generator 111 may further include an energy operating mechanism 116. Energy operating mechanism 116 may be any suitable automatic and/or user operated device in operative communication with energy generator 111 via a wired or wireless connection, such that energy operating mechanism 116 may be configured to enable activation of energy generator 111. Energy operating mechanism 116 may therefore include, but is not limited to, a switch, a push-button, or a computer. The embodiment of FIG. 2A, for example, illustrates that energy operating mechanism 116 may be a footswitch 116. Footswitch 116 may include a conductive cable coupled to an interface coupler 124 disposed on user interface 114.

Energy generator 111 may be coupled to controller 112. Controller 112 may include a processor 113 configured to receive information feedback signals, process the information feedback signals according to various algorithms, produce signals for controlling the energy generator 111, and produce signals directed to visual and/or audio indicators. For example, processor 113 may include one or more integrated circuits, microchips, microcontrollers, and microprocessors, which may be all or part of a central processing unit (CPU), a digital signal processor (DSP), an analogy processor, a field programmable gate array (FPGA), or any other circuit known to those skilled in the art that may be suitable for executing instructions or performing logic operations. That is, processor 113 may include any electric circuit that may be configured to perform a logic operation on at least one input variable. In one embodiment, for example, processor 113 may be configured to use a control algorithm to process an impedance feedback signal and general control signals for energy generator 111.

More particularly, controller 112 may be configured to perform closed loop control of energy delivery to energy delivery device 120 based on the measurement of impedance of targeted tissue sites. That is, energy delivery system 100 may be configured to measure impedance of targeted tissue sites, determine an impedance level that corresponds to a desired temperature, and supply power to energy delivery device 120 until a desired impedance level is reached. For a discussion on how impedance level correlates to temperature level, see U.S. Patent Application Publication 2009/0030477, titled SYSTEM AND METHOD FOR CONTROLLING POWER BASED ON IMPEDANCE DETECTION, SUCH AS CONTROLLING POWER TO TISSUE TREATMENT DEVICES, published on Jan. 29, 2009, which is incorporated by reference herein in its entirety. Energy delivery system 100 may also be configured to supply power to energy delivery device 120 in order to maintain a desired level of energy at the target tissue site based on impedance measurements.

Energy delivery system may further be configured to control power output from energy generator 111 in order to maintain the impedance at a level that is less than an impedance at an initial or base level (e.g., when power is not applied to the electrodes or at time to when power is first applied to a target tissue, such as at the beginning of the first pulse). The impedance may initially be inversely related to the temperature of the tissue before the tissue begins to ablate or cauterize. As such, the impedance may initially drop during the beginning of a treatment cycle and continues to fluctuate inversely relative to the tissue temperature. Accordingly, controller 112 may be configured to accurately adjust the power output from energy generator 111 based on impedance measurements to maintain a desired impedance level, and thus the temperature in a desired range.

In an alternative embodiment, processor 113 may be configured to process a temperature feedback signal via a control algorithm and general control signals for energy generator 111. Further alternative or additional control algorithms and system components that may be used in conjunction with processor 111 may be found in U.S. Pat. No. 7,104,987 titled CONTROL SYSTEM AND PROCESS FOR APPLICATION OF ENERGY TO AIRWAY WALLS AND OTHER MEDIUMS, issued Sep. 12, 2006, and in U.S. Patent Application Publication No. 2009/0030477 titled SYSTEM AND METHOD FOR CONTROLLING POWER BASED ON IMPEDANCE DETECTION, SUCH AS CONTROLLING POWER TO TISSUE TREATMENT DEVICES, published on Jan. 29, 2009, each of which is incorporated by reference herein in its entirety.

Controller 112 may additionally be coupled to and in communication with user interface 114. The embodiment of FIG. 2A illustrates that controller 112 may be electrically coupled to user interface 114 via a wire connection. In alternative embodiments, however, controller 112 may be in wireless communication with user interface 114. User interface 114 may be any suitable device capable of providing information to an operator of the energy delivery system 100. Accordingly, user interface 114 may be configured to operatively couple to each of the components of energy delivery system 100, receive information signals from the components, and output at least one visual or audio signal to a device operator in response to the information received. The surface of user interface 114 may therefore include, but is not limited to, at least one switch 122, a digital display 118, visual indicators, audio tone indicators, and/or graphical representations of components of the energy delivery system 119, 121. Embodiments of user interface 114 may be found in U.S. Patent Application Publication No. 2006/0247746 A1 titled CONTROL METHODS AND DEVICES FOR ENERGY DELIVERY, published Nov. 2, 2006, which is incorporated by reference herein in its entirety.

User interface 114 may be coupled to energy delivery device 120. The coupling may be any suitable medium enabling distribution of energy from energy generator 111 to energy deliver device 120, such as, for example, a wire or a cable 117. As illustrated in FIG. 2A, cable 117 may be connected to user interface 114 via a coupler 126 and connector 125. Energy delivery device 120 may include an elongate member 130 having a proximal portion 134 and a distal portion 132. Elongate member 130 may be any suitable longitudinal device configured to be inserted into a cavity and/or passageway of a body. Elongate member 130 may further include any suitable stiff or flexible material configured to enable movement of energy delivery device 120 through a cavity and/or passageway in a body. In one embodiment, for example, elongate member 130 may be sufficiently flexible to enable elongate member 130 to conform to the cavity and/or passageway through which it is inserted.

Elongate member 130 may be any suitable size, shape, and or configuration such that elongate member 130 may be configured to pass through a lumen 181 of an access device 180. As illustrated in FIG. 2B, access device 180 may be any suitable elongate member known to those skilled in the art having an atraumatic exterior surface 182 and configured to allow for passage of at least a portion of energy delivery device 120. In one embodiment, for example, access device 180 may be a bronchoscope. Access device 180 may include a plurality of internal channels 128, 129 extending therethrough. Internal channels 128, 129 may be configured for the passage of a variety of surgical equipment, including, but not limited to, imaging devices and tools for irrigation, vacuum suctioning, biopsies, and drug delivery. In the embodiment of FIG. 2B, for example, internal channels 128, 129 may facilitate passage of optical light fibers and/or a visualization apparatus.

Elongate member 130 may be solid or hollow. Similar to access device 180, elongate member 130 may include one or more lumens or internal channels 147 for the passage of an actuation/pull wire 146 and/or a variety of surgical equipment, including, but not limited to, imaging devices and tools for irrigation (e.g., cooling fluid), vacuum suctioning, biopsies, and drug delivery. Elongate member 130 may further include an atraumatic exterior surface having a rounded shape and/or coating. The coating be any coating known to those skilled in the art enabling ease of movement of energy delivery device 120 through access device 180 and a passageway and/or cavity within a body. The coating may therefore include, but is not limited to, a lubricious coating and/or an anesthetic.

FIGS. 2A and 2B further illustrate that an energy emitting portion 140 may be attached to distal portion 132 of elongate member 130. Energy emitting portion 140 may be permanently or removably attached to distal portion 132 of elongate member. In one embodiment, for example, energy emitting portion 140 may be permanently or removably attached to elongate member 130 via a flexible junction enabling movement of energy emitting portion 140 relative to distal portion 132 of elongate member 130. Embodiments of a junction may be found, for example, in U.S. Patent Application Publication No. 2006/0247618 A2 titled MEDICAL DEVICE WITH PROCEDURE IMPROVEMENT FEATURES, published Nov. 2, 2006, which is incorporated by reference herein in its entirety.

Energy emitting portion 140 may be any suitable device configured to emit energy from energy generator 111. In addition, as illustrated in FIG. 2C, energy emitting portion 140 may include at least one contact region 145 that may be configured to contact tissue within a cavity and/or passageway of a body. The contact region 145 may include at least a portion that is configured to emit energy from energy generator 111. Energy emitting portion 140 may further be a resilient member configured to substantially maintain a suitable size, shape, and configuration that corresponds to a size of a cavity and/or passageway in which energy delivery device 120 is inserted.

In one embodiment, for example, energy emitting portion 140 may be an expandable member. The expandable member may include a first, collapsed configuration (not shown) and a second, expanded configuration (FIG. 2B). The expandable member may include any size, shape, and/or configuration, such that in the second, expanded configuration, the contact region 145 may be configured to contact tissue in a cavity and/or passageway of a body. The expandable member of energy emitting portion 140 may be any suitable expandable member known to those skilled in the art including, but not limited to, a balloon or cage. In one embodiment, as illustrated in FIG. 2B, energy emitting portion 140 may include an expandable basket having a plurality of legs 142.

The plurality of legs 142 may be configured to converge at an atraumatic distal tip 138b of energy delivery device 120. Distal tip 138b may include a distal sleeve attached to a distal alignment retainer 144b. A distal end of each of the plurality of legs 142 may be configured to attached to distal alignment retainer 144b. In addition, the plurality of legs 142 may be configured to converge at distal portion 132 of elongate member 130 at a proximal sleeve 138a and a proximal alignment retainer 144a. Proximal alignment retainer 144a may be configured to be removably or fixedly attached to distal portion 132 of elongated body 130 and attached to a proximal end of each of the plurality of legs 142. Each of the distal and proximal alignment retainers 144a, 144b may be configured to maintain each of the plurality of legs 142 a predetermined distance apart from one another. Additional or alternative features of distal and/or proximal alignment components 144a, 144b may be found, for example, in U.S. Pat. No. 7,200,445, titled ENERGY DELIVERY DEVICES AND METHODS, issued on Apr. 3, 2007, which is incorporated by reference herein in its entirety.

Energy emitting portion 140 may further include at least one electrode. The at least one electrode may be any suitable electrode known to those skilled in the art and configured to emit energy. The at least one electrode may be located along the length of at least one of the plurality of legs 142 and may include at least a portion of the contact region of energy emitting portion 140. Accordingly, the at least one electrode may include, but is not limited to, a band electrode or a dot electrode. Alternatively, the embodiment of FIGS. 2A-C illustrates that at least one leg 142 of the energy emitting portion is made up of a single, elongate electrode (FIG. 2C). In one embodiment, for example, the elongate electrode may include electrical insulator material 143 covering a proximal portion and/or a distal portion of the elongate electrode (FIG. 2C). In addition, at least a portion 145 of the electrode may be exposed, forming the active/contact region for delivering energy to tissue.

As illustrated in FIGS. 2A-2B, each of the plurality of legs 142 of energy emitting portion may be configured to form an expandable basket-type shape when in the second, expanded configuration. Accordingly, upon expansion of energy emitting portion 140, each of the plurality of legs 142 may be configured to bow radially outward, in the direction of arrow O, from a longitudinal axis of energy delivery device 120 as wire 142 moves proximally in the direction of arrow P. Energy emitting portion 140 may further be configured to return to the first, collapsed configuration upon release of wire 146, which may thereby cause each of the plurality of legs 142 to move radially inward in the direction of arrow I.

The at least one electrode may be monopolar or bipolar. The embodiment of FIG. 2A illustrates an energy emitting portion 140 including monopolar electrodes. Accordingly, the embodiment of FIG. 2A further includes a return electrode component configured to complete an electrical energy emission or patient circuit between energy generator 111 and a patient (not shown). The return electrode component may include a conductive pad 115, a coupler 123 coupled to user interface 114 and a conductive cable extending between and in electrical communication with conductive pad 115 and proximal coupler 123. Conductive pad 115 may include a conductive adhesive surface configured to removably stick to a patient's skin. In addition, conductive pad 115 may include a surface area having a sufficient size in order to alleviate burning or other injury to the patient's skin that may occur in the vicinity of the conductive pad 115 during energy emission.

Energy delivery device 120 may further include a handle 150. Handle 150 may be any suitable handle known to those skilled in the art configured to enable a device operator to control movement of energy delivery device 120 through a patient. In addition, in some embodiments, handle 150 may further be configured to control expansion of energy emitting portion 140. Handle 150 may accordingly include an actuator mechanism, including, but not limited to, a squeeze handle, a sliding actuator, a foot pedal, a switch, a push button, a thumb wheel, or any other known suitable actuation device.

FIG. 2A illustrates an example of a handle 150 according to an embodiment of the present disclosure. Handle 150 may be configured such that a single operator can hold access device 180 in one hand (e.g., a first hand) and use the other hand (e.g., a second hand) to both (a) advance elongated body 130 and energy emitting portion 140 through lumen 181 of access device 180 until energy emitting portion 140 extends beyond the distal end of access device 180 and is positioned at a desired target site and (b) pull wire 146 to move each of the plurality of legs 142 radially outward until they contact tissue, while elongate member 130 is held in place relative to access device 180 with the same second hand. The same device operator can also operate energy operating mechanism 116, such that the entire procedure can be performed by a single person.

As illustrated in FIG. 2A, handle 150 may include a first portion 151 and a second portion 152 movably coupled to first portion 151. The movable coupling may be any suitable mechanism known to those skilled in the art that may be configured to enable second portion 152 to move relative to first portion 151. In one embodiment, for example, second portion 152 may be rotatably coupled to first portion 151 by a joint 153. Handle 150 may further be connected to wire 146 such that movement of second portion 152 relative to first portion 151 may be configured to cause energy emitting portion 140 to transition between the first, collapsed configuration and the second, expanded configuration.

First and second portions 151, 152 may be configured to form a grip 154 and a head 156 located at an upper portion of the grip 154. The head 156, for example, can project outwardly from the grip such that a portion of the grip 154 is narrower than the head 156. Head 156 and grip 154 may be any suitable shape known to those skilled in the art such that a device operator can hold handle 150 in one hand. For example, the embodiment of FIG. 2A illustrates that first portion 151 may include a first curved surface 161 with a first neck portion 163 and a first collar portion 165, and second portion 152 may include a second curved surface 162 with a second neck portion 164 and a second collar portion 166. First and second curved surfaces 161, 162 may be configured such that they are arranged to define a hyperbolic-like shaped grip 154 when viewed from a side elevation.

Energy delivery device 120 may further include at least one sensor (not shown) configured to be in wired or wireless communication with the display and/or indicators on user interface 114. The at least one sensor may be configured to sense tissue temperature and/or impedance level. In one embodiment, for example, energy emitting portion 140 may include at least one impedance sensor and/or at least one temperature sensor in the form of a thermocouple. Embodiments of the thermocouple may be found in U.S. Patent Application Publication No. 2007/0100390 A1 titled MODIFICATION OF AIRWAYS BY APPLICATION OF ENERGY, published May 3, 2007, which is incorporated by reference herein in its entirety.

In addition, the at least one sensor may be configured to sense functionality of the energy delivery device. That is, the at least one sensor may be configured to sense the placement of the energy delivery device within a patient, whether components are properly connected, whether components are properly functioning, and/or whether components have been placed in a desired configuration. In one embodiment, for example, energy emitting portion 140 may include a pressure sensor or strain gauge for sensing the amount of force energy emitting portion 140 exerts on tissue in a cavity and/or passageway in a patient. The pressure sensor may be configured to signal energy emitting portion 140 has been expanded to a desired configuration such that energy emitting portion 140 may be prevented from exerting a damaging force on surrounding tissue or on itself (e.g., electrode inversion). In addition, or alternatively, the pressure sensor may be configured to signal that not enough force has been exerted, which may thereby indicate that further contact may be needed between energy emitting portion 140 and the surrounding tissue. Accordingly, the at least one sensor may be placed on any suitable portion of energy delivery device including, but not limited to, on energy emitting portion 140, elongate member 130, and/or distal tip 138b.

Energy delivery device 120 may include at least one imaging or mapping device (not shown) located on one of the energy emitting portion 140, elongate member 130, and/or distal tip 138b. The imaging or mapping device may include a camera or any other suitable imaging or mapping device known to those skilled in the art and configured to transmit images to an external display. Energy delivery device 120 may additionally include at least one illumination source. The illumination source may be integrated with the imaging device or a separate structure attached to one of the energy emitting portion 140, elongate member 130, access device 180, and/or distal tip 138b. The illumination source may provide light at a wavelength for visually aiding the imaging device. Alternatively, or in addition, the illumination source may provide light at a wavelength that allows a device operator to differentiate tissue that has been treated by the energy delivery device 120 from tissue that has not been treated.

Additional embodiments of the imaging or mapping device may be found in U.S. Patent Application Publication Nos. 2006/0247617 A1 titled ENERGY DELIVERY DEVICES AND METHODS, published Nov. 2, 2006; 2007/0123961 A1 titled ENERGY DELIVERY AND ILLUMINATION DEVICES AND METHODS, published May 31, 2007; and 2010/0268222 A1 titled DEVICES AND METHODS FOR TRACKING AN ENERGY DEVICE WHICH TREATS ASTHMA, published Oct. 21, 2010, each of which are incorporated by reference herein in its entirety.

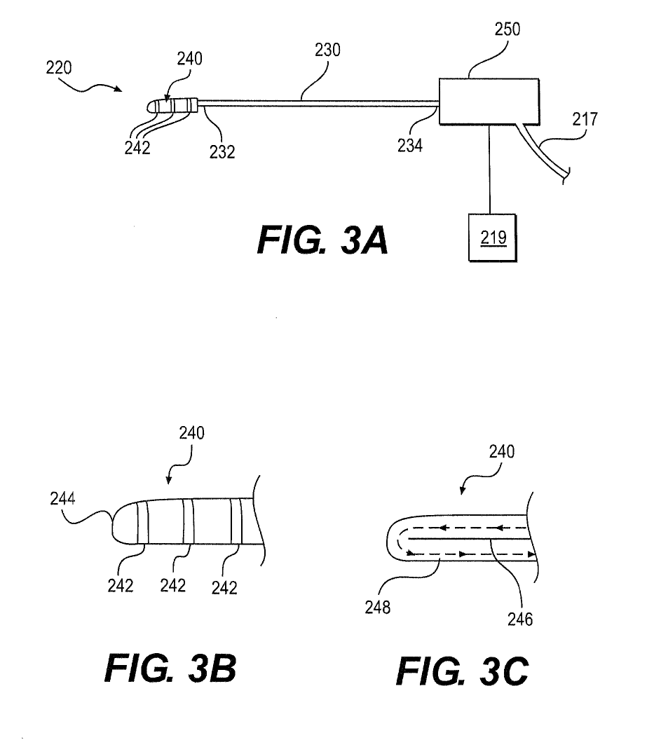

FIG. 3A illustrates an energy delivery device 220 configured to delivery energy to tissue in a cavity and/or passageway in a body, according to a second embodiment of the present disclosure. Similar to energy delivery device 120 of FIG. 2A, energy delivery device 220 may be sized such that it may be delivered into a body via lumen 181 in access device 180. In addition, energy delivery device 220 may be configured to couple to user interface 114 via any suitable medium configured to enable distribution of energy from energy generator 111 to energy delivery device 220, such as, for example, a conductive wire or cable 217. Conductive wire or cable 217 may be configured to connect to user interface 114 via the coupler 126 and connector 125 of FIG. 2A.

Energy delivery device 220 may further include an elongate member 230 having a proximal end 234 and a distal end 232. Elongate member 230 may be any suitable longitudinal device configured to be inserted into a cavity and/or passageway in a body and may include features similar to elongate member 130 of FIG. 2A. For example, elongate member 230 may include any suitable material configured to enable movement of energy delivery device 220 through a cavity and/or passageway in a body. In addition, elongate member 230 may be solid or hollow and may include one or more lumens or internal channels (not shown) for the passageway of a variety of surgical equipment. Elongate member 230 may also include an atraumatic exterior surface (e.g., rounded). The exterior surface may also include a material, including, but not limited to, a lubricant or an anesthetic.

Energy delivery device may further include a handle 250 attached to proximal end 234 of elongate member 230. Handle 250 may be removably or permanently attached to elongate member 230. In addition, handle 250 may be any suitable shape, size, and/or configuration such that a device operator may be able to grip handle 250 in one hand and use handle 250 to advance energy delivery device 220 through lumen 181 of access device 180.

As illustrated in FIG. 3A, elongate member 230 may further be attached to an energy emitting portion 240 at its distal end 232. Similar to energy emitting portion 140 of FIG. 2A, energy emitting portion 240 may be permanently or removably attached to elongate member 230. Energy emitting portion 240 may further be directly attached to elongate member 230. Alternatively, energy emitting portion 240 may be indirectly attached to elongate member 230 via a connecting means, such as, for example, a flexible junction that may be configured to enable movement of energy emitting portion 240 relative to elongate member 230.

Energy emitting portion 240 may be any suitable device configured to emit energy from energy generator 111. In the embodiment of FIG. 3A, for example, energy emitting portion 240 may be a cooled electrode device 240. Generally, cooled electrode devices have been used to ablate large volumes of cardiac or tumor (e.g., liver) tissue, where relatively greater tissue damage and/or high temperatures may be required. Use of cooled electrode device 240 in the airways of a lung, however, may be beneficial due to its ability to maintain an electrode temperature below 100 degrees Celsius in order to prevent early impedance roll-off due to the formation of micro-bubbles on tissue within an airway. Another benefit of using cooled electrode device 240, for example, may include protecting surface tissue by leaving it unaffected while simultaneously treating underlying tissue. This benefit may be realized even at temperatures below 100 degrees Celsius.

Cooled electrode device 240 may be any suitable size, shape, and/or configuration known to those skilled in the art such that cooled electrode device 240 may be capable of movement through an airway of a lung. In addition, cooled electrode device 240 may be sized, shaped, and configured to contact walls of an airway in a lung. FIG. 3B illustrates a cooled electrode device 240 according to an embodiment of the present disclosure. Cooled electrode device 240 may be an elongate member with an atraumatic outer surface 244, such that cooled electrode device 240 may be configured to move through an airway of a lung without causing unwanted or collateral damage to tissue (e.g., inner lumen of airway, such as epithelium, pulmonary blood vessels, airway smooth muscle, nerves, etc.). Accordingly, outer surface 244 of cooled electrode device 240 may include a material to aid in movement, such as a lubricant and/or an anesthetic. Exemplary cooled electrode devices are described in U.S. Pat. No. 7,949,407, which is incorporated herein by reference in its entirety.

Cooled electrode device 240 may further include at least one electrode 242 on its outer surface 244 that may be configured to apply energy to tissue in a passageway and/or cavity (e.g., an airway in a lung). The at least one electrode 242 may be any suitable electrode known to those skilled in the art, including, but not limited to, an elongate electrode or a ring or dot electrode. The embodiment of FIG. 3B illustrates that the at least one electrode 242 may be a band electrode, which may or may not substantially surround the circumference of cooled electrode device 240.

Moreover, FIG. 3B illustrates that cooled electrode device 240 may include a hollow inner portion 248 and a partition 246 that may be configured to allow the internal circulation of a cooling fluid. The cooling fluid may be any suitable fluid known to those skilled in the art (e.g., cooled saline) and configured to cool the tissue and/or electrode before, during, or after energy delivery by the at least one electrode 242 in order to prevent undesired effects at the electrode/tissue interface (e.g., unwanted tissue damage and/or impedance roll off due to the formation of micro-bubbles). Accordingly, the cooled fluid may include, but is not limited to, water and saline solution. FIG. 3A illustrates that the cooling fluid may be configured to circulate through cooled electrode device 240 with the help of a cooling fluid source 219 that may be connected, via any suitable connection means known to one skilled in the art, to energy delivery device 220.

Energy delivery device 220 may further include features similar to those disclosed in relation to energy delivery device 120 of FIG. 2A. For example, energy delivery device 220 may include at least one sensor (not shown) configured to sense tissue impedance level and/or tissue temperature and configured to be in wired or wireless communication with the display and/or indicators on user interface 114. In addition, the at least one sensor may be configured to sense functionality of energy delivery device 220, which may include, but is not limited to, connection, placement, pressure, and functioning sensing of energy delivery device 220. Accordingly, the at least one sensor may be placed on any suitable portion of energy delivery device 220 including, but not limited to, on cooled electrode device 240, handle 250, and elongate member 230. In addition, similar to energy delivery device 120 of FIG. 2A, energy delivery device 220 may include at least one imaging or mapping device and/or at least one illumination source located on at least one of cooled electrode 240, handle 250, and elongate member 230.

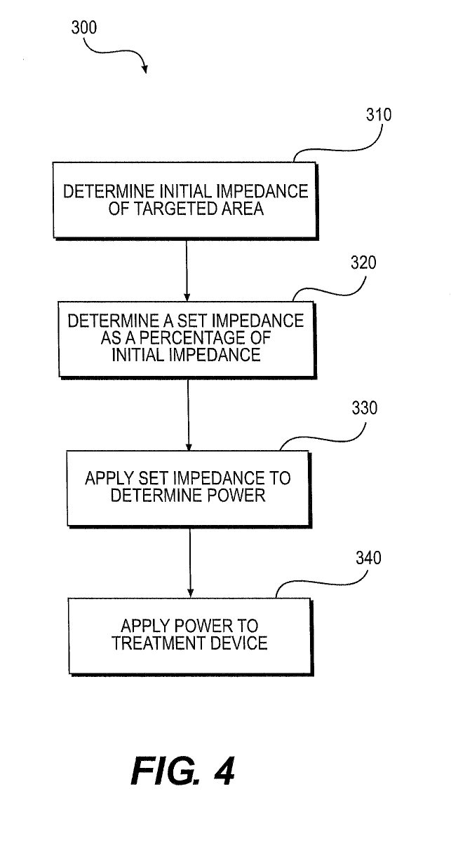

FIG. 4 illustrates a flow diagram of a method for controlling power during treatment 300 based on impedance measurements using the cooled energy delivery device 220 of FIG. 3A. During treatment of tissue within the lung of an airway, for example, it is important to accurately measure maximum tissue temperature in order to determine the appropriate amount of energy delivery for treatment of the tissue. As illustrated in FIG. 3A, energy delivery device employs a cooled electrode device 240. Cooled electrode device 240 may be configured to enable more current to be driven into the tissue than a non-cooled electrode, which may thereby move the maximum tissue temperature away from the electrode/tissue interface and into the tissue. Accordingly, when cooled electrode device 240 is employed, measurement of temperature at the electrode/tissue interface may not be an accurate measure of maximum tissue temperature. It has been determined, however, that impedance level measurements in the tissue indirectly correspond to/measure maximum tissue temperature of a volume of tissue, and not the temperature at the electrode/tissue interface. Using impedance measurements to control power to a cooled electrode device, therefore, may be a superior way to control tissue treatment than temperature monitoring (which is limited by temperature measurement at the electrode/tissue interface).

The method illustrated in FIG. 4, which controls power during treatment based on impedance measurements of tissue, as opposed to temperature measurements of tissue, may have the following advantages. Impedance control may enable the same volume of tissue to be ablated as with temperature control while producing a lower maximum tissue temperature. In addition, the level of damage produced during impedance control may only depend on a variable of measured impedance, whereas the level of damage produced by temperature control may depend on two variables, temperature and amount of cooled electrode device cooling.

Moreover, typical temperature-controlled devices generally measure tissue temperature at the electrode-tissue interface. The temperature at the electrode-tissue interface is generally the maximum temperature experienced by the tissue. By maintaining the electrode-tissue interface temperature for a pre-determined period of time, the treatment effect within the tissue may be predicted. To increase the effect of a particular treatment, the temperature at the electrode-tissue interface or the treatment time would need to be increased. For cooled electrodes, however, where the tissue temperature sensor may be isolated from the electrode temperature, the treatment effect may be a function of both the treatment temperature as well as the cooled electrode temperature. That is, altering either the treatment temperature or the cooled electrode temperature could change the treatment effect. Impedance control, on the other hand, allows the treatment effect to be a function of only the control impedance and the duration of the treatment, regardless of the temperature at the cooled electrode.

Further, impedance control may be configured to lower cost and complexity of both energy generator 111 and energy delivery device 220, relative to use of energy delivery device 120, because there is no need for temperature sensors (e.g., thermocouples).

FIG. 4 illustrates that the method for controlling power during treatment 300 based on impedance measurements using the energy delivery device 220 may first include a step 310 of determining an initial impedance of tissue at a targeted treatment site. In one embodiment, for example, the initial impedance may be based on an initial measurement of voltage or current at body temperature of the tissue and/or of energy delivery device 220. Alternatively, the initial impedance may be determined based on a test or pre-treatment low energy pulse (i.e., a non-therapeutic energy pulse that does not heat tissue) at the targeted treatment site while keeping the power or current constant.

The method 300 may further include a step 320 of determining a desired or set impedance that correlates to a desired treatment temperature or temperature range. In some embodiments, set impedance may be determined as a percentage of the initial impedance. Alternatively, the set impedance may be based on parameters of the targeted treatment site (e.g., size of the passageway, initial temperature of the passageway, mucus or moisture content of the passageway, or other physiologic factors), parameters of energy delivery device 220 (e.g., configuration or geometry of cooled electrode, such as electrode 242 spacing, length, width, thickness, radius), the desired temperature range, parameters of a test or pre-treatment pulse, and/or other parameters associated with the effect of energy on the tissue (e.g., bipolar or monopolar energy delivery). These parameters may be automatically detected from the initial impedance value or may be measured via a sensor (e.g., a device mounted sensor, a non-contact infrared sensor, and/or a standard thermometer to measure an initial temperature of the passageway). Accordingly, method 300 may include a step 330 of applying the set impedance to an algorithm, such as a PID algorithm, to determine the power to be applied to an energy delivery device. Further details with respect to the calculation of set impedance and/or the PID algorithm can be found in U.S. Patent Application Publication 2009/0030477, titled SYSTEM AND METHOD FOR CONTROLLING POWER BASED ON IMPEDANCE DETECTION, SUCH AS CONTROLLING POWER TO TISSUE TREATMENT DEVICES, published Jan. 29, 2009, which is incorporated by reference herein in its entirety.

Method 300 may further include periodically measuring current or present impedance values during treatment and applying the measured impedance values to the algorithm to control the power needed to achieve, return to, or maintain the desired impedance and/or temperature. For example, during treatment, energy delivery system may identify a present impedance level as being higher that the set impedance level, and use both the present and set impedance levels as inputs into the PID algorithm to determine the power level outputted by cooled electrode device 240. Method 300 may then continue with a step 340 of delivering energy to the tissue 340 with the cooled electrode device 240 in a manner that maintains a desired temperature of the tissue at the targeted treatment site.

Alternatively, or in addition, energy delivery system may periodically or continuously perform some or all of the steps of method 300 of FIG. 4. For example, in one embodiment, the energy delivery system may continuously determine the set impedance during a treatment, and adjust power levels based on any changes in the set impedance. Alternatively, the energy delivery system may periodically determine the set impedance, and may adjust power levels based on a set impedance change being above a certain threshold change. Moreover, the energy delivery system may recalculate the set impedance between treatments. For example, after a treatment at a first targeted treatment site, energy delivery device may move to a second targeted treatment site, calculate a new set impedance, and adjust the applied power output accordingly.

Furthermore, while the devices disclosed herein may use a constant current, pre-treatment pulse to determine control impedance, those of ordinary skill in the art will readily recognize that a constant power or constant voltage pulse may also be used.

Other embodiments of the present disclosure will be apparent to those skilled in the art from consideration of the specification and practice of the present disclosure disclosed herein. It is intended that the specification and examples be considered as exemplary only, with a true scope and spirit of the present disclosure being indicated by the following claims.

What is claimed is:

1. An energy delivery system, comprising:
an energy generator;
a cooled electrode device; and
a controller connected to the energy generator and including a processor;
wherein the controller is configured to control power output by the cooled electrode device based on a measured impedance level of tissue at a target treatment site.

2. The energy delivery system of claim 1, wherein the controller is configured to control power output based on a second impedance level set in the controller.

3. The energy delivery system of claim 2, wherein the controller is configured to calculate the second impedance level.

4. The energy delivery system of claim 3, wherein the controller is configured to calculate the second impedance level based on a percentage of the an initial impedance level measured at the target treatment site.

5. The energy delivery system of claim 3, wherein the controller is configured to calculate the second impedance level based on at least one of: a parameter of tissue at the target treatment site, a parameter of the cooled electrode device, a desired temperature range of tissue at the target treatment site, and a parameter of a pre-treatment energy output pulse.

6. The energy delivery system of claim 1, wherein the controller is configured to determine a temperature that correlates to the measured impedance level.

7. The energy delivery system of claim 1, wherein the cooled electrode device includes an internal portion for cooling the cooled electrode device when the cooled electrode device is in contact with tissue at the target treatment site.

8. A method for treating tissue, comprising:
determining an initial impedance level of tissue at a targeted treatment site with an energy delivery system comprising an energy generator, a cooled electrode device, and a controller including a processor;
determining a second impedance level with the energy delivery system, wherein the second impedance level corresponds to a desired temperature of tissue at the targeted treatment site; and
applying power to the tissue at the targeted treatment site through the cooled electrode device, wherein a power output level is determined based on the second impedance level.

9. The method of claim 8, wherein the tissue at the targeted treatment site is located within an airway in a lung of a body.

10. The method of claim 8, wherein the controller determines the second impedance level.

11. The method of claim 10, wherein the controller determines the second impedance level based on a percentage of the initial impedance level.

12. The method of claim 10, wherein the controller determines the second impedance level based on at least one of: a parameter of tissue at the target treatment site, a parameter of the cooled electrode device, a desired temperature range of tissue at the target treatment site, and a parameter of a pre-treatment energy output pulse.

13. The method of claim 8, wherein the controller determines the power output level.

14. The method of claim 13, wherein the determination of the power output level includes applying the second impedance level to a PID algorithm.

15. The method of claim 8, further including repeating the step of determining the second impedance level throughout a cycle of treating tissue at the targeted treatment site.

16. The method of claim 15, further including adjusting the power output level based the re-determined second impedance level.

17. The method of claim 8, wherein the targeted treatment site is a first targeted treatment site, and wherein the method includes determining a second impedance level at a second treatment site, and applying power to the second treatment site based on the second impedance level determined at the second treatment site.

18. The method of claim 8, further comprising the step of cooling the tissue before, during, or after the step of applying power to the tissue.

19. An energy delivery system, comprising:
an energy delivery device including a cooled electrode device configured for connecting to an energy generator and a controller;
wherein the cooled electrode device is configured to output power based on (a) an initial impedance level of tissue at a targeted treatment site and (b) a second impedance level corresponding to a desired temperature of tissue at the targeted treatment site.

20. The energy delivery system of claim 19, the cooled electrode device is configured to output power based on an application of the second impedance level to a PID algorithm
